(19) United States
(12) Patent Application Publication
(10) Pub. No.: US 2010/0297159 A1
(43) Pub. Date: Nov. 25, 2010

(54) THOMSEN-FRIEDENREICH DISACCHARIDE MODIFIED IMMUNOGEN (TFD), PREPARATION PROCEDURE, COMPOSITIONS COMPRISING IT, USES, AND TREATMENT METHODS

(75) Inventors: Fernando José Irazoqui, Cordoba (AR); Víctor Germán Sendra, Cordoba (AR); Gustavo Alejandro Nores, Cordoba (AR)

Correspondence Address: THE NATH LAW GROUP, 112 South West Street, Alexandria, VA, 22314, US

(73) Assignees: CONSEJO NACIONAL DE INVESTIGACIONES CIETIFICAS Y TECNICAS (CONICET), Buenos Aires (AR); UNIVERSIDAD NACIONAL DE CORDOBA, Cordoba (AR); INIS BIOTEC LLC, Milford, DE (US)

(21) Appl. No.: 12/743,956

(22) PCT Filed: Nov. 19, 2008
(86) PCT No.: PCT/IB08/54850

§ 371 Date: Aug. 3, 2010

(30) Foreign Application Priority Data
Nov. 20, 2007 (AR) P070105146

(51) Int. Cl. A61K 39/00; C07K 9/00; C13K 13/00; A61P 35/00; A61P 37/04

(52) U.S. Cl. 424/185.1; 530/322; 536/123.13

(57) ABSTRACT

Modified Thomsen-Friedenreich disaccharide (TFD) immunogen, preparation procedure, compositions containing it, uses, and treatment methods. More specifically, the present invention refers to a immunogen obtained by modifying TFD, and comprising the general formula D-TFD-Lys(n)-C, wherein D is a hydrophobic terminal residue, TFD is disaccharide Galβ3GalNAcα; Lys(n) is a lysine connector, and n is an integer between 1 and 5, and C is a carrier. In a preferred embodiment, the modified invention immunogen comprises the general formula BzlαTFD-Lys5-KLHs, wherein Bzl is benzyl, TFD is disaccharide Galβ3GalNAcα; Lys5 is penta-lysine, and KLHs is succinilated Keyhole limpets hemocyanin.

The present invention relates to a modified Thomsen-Friedenreich disaccharide immunogen (TFD), a preparation procedure, compositions containing it, uses, and treatment methods. More specifically, the present invention relates to a Thomsen-Friedenreich disaccharide modified immunogen (TFD), comprising general formula D-TFD-Lys(n)-C, wherein D is a hydrophobic terminal residue, TFD is disaccharide Galβ3GalNAcα; Lys(n) is a lysine connector and n is an integer between 1, and 5, and C is a carrier. In a preferred embodiment, the modified invention immunogen comprises general formula BzlαTFD-Lys5-KLHs, wherein Bzl is benzyl, TFD is disaccharide Galβ3GalNAcα; Lys5 is a penta-lysine and KLHs is succinilated Keyhole limpets hemocyanin.

### BACKGROUND

Tumor cell phenotype is highly influenced by the presence of complex glycans such as glycoproteins, glycolipids, and proteoglycans. Oligosaccharide chains are involved in diverse cell protection mechanisms, both physical and biochemical, and more significantly in specific acknowledgement events. Mucin-type O-glycane chains are aberrantly synthesized on epithelial tumor cell glycoproteins (Brockhausen I, EMBO Rep. 7, 599-604, 2006), resulting in an altered cell-cell communication. O-glycane biosynthesis like Mucin-type glycane is a post-translation modification catalyzed by glycosyl transferases residing in the Golgi, an organelle functioning as a cell homeostasis sensible sensor. Frequently, epithelial tumor cell show reduced or no core2 β6GlcNAc-transferase and core3 β3GlcNAc-transferase expression activity, which are involved in the production of mucin-type O-glycanes. Suppression of these genes results in expression and secretion of mucin-type O-glycane truncated chains showing normally hidden cryptic structures (Hanisch F G, et. al., Curr. Protein Pept. Sci. 7, 307-315, 2006). Some examples are antigens Tn (GalNAcα1-Ser/Thr), and T (Galβ3GalNAcα1-Ser/Thr) (Kumar S R, et. al., Clin. Cancer Res. 11, 6868-6871, 2005). Antigen T glycidic domain (or Thomsen-Friedenreich antigen) is known as Thomsen-Friedenreich disaccharide (TFD, Galβ3GalNAcα). Antigen T and related antigenic structures are commonly exposed by breast and colon epithelial tumor cells (Goletz and col., 2003). Expression of truncated O-glycanes by tumor cell is associated with host immunesuppression (Agrawal B, et. al., Int. Immunol. 17, 391-399, 2005). High serum levels of glyco-conjugates exposing antigens T and Tn correlate directly with progression and tumoral aggressiveness (Kawaguchi T, et. al., Breast Cancer Res. Treat. 92, 223-230, 2005), and inversely with lung adenocarcinoma patient survival (Takanami I. Oncol. Rep. 6, 341-344, 1999). In metastases adhesion, residues related to antigen T play a key role in tumor cell interaction with endothelial tissue (Yu L G, et. al., J. Biol. Chem. 282, 773-781, 2007). The level of antigen T recognizing IgG-type antibodies in gastric cancer patients is significantly lower than in normal subjects. Gastric cancer patients sero-positive for Helicobacter pylori and having high level of IgG-type anti-antigen T antibodies (considered strong answerers) show a significantly higher survival ratio than weakly responding patients (Kurtenkov O, et. al., Immunol. Invest. 32, 83-93, 2003).

Glycane engineering is an emerging are of biomedical investigation, which shows multiple applications (Allison D D, et. al., Tissue Eng. 12, 2131-2140, 2006). The main objective of cancer vaccine design is the creation of specific immunogens from very well characterized antigens, and TFD is an attractive molecule for such purpose (Irazoqui F J, et. al. Immunol. Cell Biol. 83, 405-412, 2005). Self embryogenic antigens of low molecular weight and glycidic nature, such as antigen T, are scarcely immunogenic. Tests performed generate an important immune response using TFD alone have been fruitless. Immunigenic glycane design requires several components with different roles. Generally, immune response to glycanes is independent of T cells, producing isotype IgM. Inclusion of a carrier protein in an immunogen modifies the type of immune response towards T cell dependence, producing isotype IgG with immunologic memory.

In order to make TFD more immunogenic, carriers, such as KLH (Keyhole limpets hemocyanin), which eliminates immunologic tolerance to self embryogenic antigens and terminal residues such as benzyl or p-nitrophenol were added (Irazoqui F J, et. al., Mol. Immunol. 38, 825-831, 2002, and Irazoqui F J, et. al., Glycobiology 10, 781-787, 2000). However, none of these configurations have achieved the generation of high immunogenicity levels. In this context, the immunogen generated from the TFD alfa-benzyl derivative conjugated with carrier KLHs that is BzlαTFD-KLH, has greater immunogenic capacity than the non-modified disaccharide TFD-KLH, which is incapable of generating an important title of antibodies against antigen T, due to its low immunogenicity. In addition, BzlαTFD-KLH is a better immunogen than the p-TFD nitrophenyl derivative coupled to the same carrier that is pNPαTFD-KLH, which indicates that the alfa-benzyl derivative is the best choice for the referred glycane engineering in order to obtain a greater title of anti-TFD antibodies. However, as mentioned above, it is possible to have a greater TFD immunogenicity control by making a more rigid structure (Irazoqui and col., Immunol. Cell Biol. 83, 405-412, 2005).

Most immunogens generating anti-Thomsen-Friedenreich disaccharide immune response do not recognize natural antigens (such as antigen T o Tn) associated to tumors. It is necessary to generate more rigid structure immunogens, to obtain immunogenic configurations generating adequate amount and quality of anti-antigen T, tumor-associated immune response.

### BRIEF DESCRIPTION OF THE INVENTION

It is an object of the present invention to provide a modified Thomsen-Friedenreich disaccharide (TFD) immunogen comprising general formula D-TFD-Lys(n)-C, wherein D is a hydrophobic terminal residue, TFD is disaccharide Galβ3GalNAcα; Lys(n) is a lysine connector, and n is an integer between 1 and 5, and C is a carrier. In a preferred embodiment, the modified invention immunogen comprises general formula BzlαTFD-Lys5-KLH, wherein Bzl is benzyl, TFD is disaccharide Galβ3GalNAcα; Lys5 is a penta-lysine and KLH is Keyhole limpets hemocyanin.

It is another object of the present invention to provide a procedure for preparing a modified immunogen, such procedure comprising the following stages:

a. Covalently link lysine connector Lys(n) to a carrier, wherein n is an integer between 1 and 5;

b. Oxydize molecule BzlαTFD in terminal galactose carbon 6 to generate an aldehyde in said C6;

c. Covalently link Lys(n)-C connector amine residue of stage a), and aldehyde in BzlαTFD terminal galactose C6 of stage b) by reduction amination in the presence of NaCNBH₃; and

d. Dyalize reaction product, and recover the immunogen.

It is another object of present invention to provide a composition to increase anti-tumoral immunity comprising an effective amount of the modified Thomsen-Friedenreich disaccharide (TFD) immunogen of general formula D-TFD-Lys(n)-C, wherein D is a hydrophobic terminal residue, TFD is disaccharide Galβ3GalNAcα, Lys(n) is a lysine connector and n is an integer between 1 and 5, and C is a carrier; and pharmaceutically acceptable supports and/or excipients. Such composition may be as a liquid, gel, solid, or aerosol, and may in addition comprise an adjuvant. In a preferred embodiment, the composition comprises immunogen BzlαTFD-Lys5-KLH, wherein Bzl is benzyl, TFD is disaccharide Galβ3GalNAcα; Lys5 is a penta-lysine, and KLH is Keyhole limpets hemocyanin, pharmaceutically acceptable supports and/or excipients.

It is another object of present invention to provide a composition to inhibit adhesion of tumor cells and metastases capacity comprising an effective amount of a modified Thomsen-Friedenreich disaccharide (TFD) immunogen of general formula D-TFD-Lys(n)-C, wherein D is a hydrophobic terminal residue, TFD is disaccharide Galβ3GalNAcα, Lys(n) is a lysine connector and n is an integer between 1 and 5, and C is a carrier; and pharmaceutically acceptable supports and/or excipients. Such composition may be as liquid, gel, solid, or aerosol, and may in addition comprise an adjuvant. In a preferred embodiment, the composition comprises immunogen BzlαTFD-Lys5-KLH, wherein Bzl is benzyl, TFD is disaccharide Galβ3GalNAcα; Lys5 is a penta-lysine, and KLH is Keyhole limpets hemocyanin, pharmaceutically acceptable supports and/or excipients.

It is another object of the present invention to provide the use of an modified Thomsen-Friedenreich disaccharide (TFD) immunogen comprising general formula D-TFD-Lys(n)-C, wherein D is a hydrophobic terminal residue, TFD is disaccharide Galβ3GalNAcα; Lys(n) is a lysine connector, and n is an integer between 1 and 5, and C is a carrier to prepare a medication to improve anti tumoral immunity.

It is another object of the present invention to provide the use of modified Thomsen-Friedenreich disaccharide (TFD) immunogen comprising general formula D-TFD-Lys(n)-C, wherein D is a hydrophobic terminal residue, TFD is disaccharide Galβ3GalNAcα; Lys(n) is a lysine connector and n is an integer between 1 and 5, and C is a carrier to prepare a medication for the treatment of cancer.

It is another object of the present invention to provide the use of modified Thomsen-Friedenreich disaccharide (TFD) immunogen comprising general formula D-TFD-Lys(n)-C, wherein D is a hydrophobic terminal residue, TFD is disaccharide Galβ3GalNAcα; Lys(n) is a lysine connector, and n is an integer between 1 and 5, and C is a carrier to prepare a medication to reduce metastases capacity of a tumor.

It is another object of present invention to provide a method for immunization of a mammal against antigen T, wherein such method comprises administering an effective amount of a composition, wherein composition comprises a modified Thomsen-Friedenreich disaccharide (TFD) immunogen of general formula D-TFD-Lys(n)-C, wherein D is a hydrophobic terminal residue, TFD is disaccharide Galβ3GalNAcα, Lys(n) is a lysine connector, and n is an integer between 1 and 5, and C is a carrier; and pharmaceutically acceptable supports and/or excipients, wherein the mammal may carry a cancer. Treatment method may be applied jointly with other immunity increasing compounds.

### DESCRIPTION OF FIGURES

FIG. 1 shows TFD engineering to reduce glycan flexibility based on theoretical studies. This figure shows antigen T MUC1 [GS (Galβ3GalNAcα)TAP] (A) Gal-O-GalNAc torsion angles BzlαTFD (B), TFD-Lys5 (C), and BzlαTFD-Lys5 (D) illustrated Ramachandran diagrams. Torsion angles φ (O5-C1-O-C3′), and ψ (C1-O-C3′-C2′) refer to rotations around links C1-O, and O-C3′, respectively. Scales adjacent to each graph represent energy values (kcal/mol) for low (red), and high (violet) energy conformers.

FIG. 2 illustrates glycoconjugated synthesis control. Lys5 (bottom), and BzlαTFD-Lys5 (top) molecular weights were determined by MALDI-TOF mass spectrometry, showing BzlαTFD with Lys5 covalent link (A). Invention immunogen (B) synthesis, that is, BzlαTFD to Lys5-KLHs covalent link was demonstrated by enzyimy-lectin assay (ELA) against Agaricus bisporus lectin. Multi-well polystirene plates were covered with ASG (filled square), BzlαTFD-Lys5-KLHs (white circle), Lys5-KLHs (inverted white triangle), KLHs (filled circle), and BSA (white square) in PBS overnight at 4° C., and were then incubated with diverse ABL-HRP concentrations for 2 hours at room temperature. A color reaction developed, and absorbance was determined.

FIG. 3 shows fluorescent marking of epithelial tumor cells using antibodies generated by invention immunogen BzlαTFD-Lys5-KLHs. Cytological immunomarking of three tumor cell lines (HT29, MCF7y T47D), and human mononuclear cells were assessed (HMC). To inhibit cell-antibody marking, sera were pre-incubated with BzlαGalNAc o Bzlo(TFD (50 mM) in PBS for 1 hour at room temperature. Samples were washed with PBS, and incubated with mouse anti-immunoglobulins against rabbit antibodies conjugated to Alexa488 ( 1/1000) for 1 hour at room temperature. Fluorescent marked cells were examined with Zeiss fluorescent microscope.

FIG. 4 shows cell adhesion assays. Multi-well plates were covered with carbohydrate linking proteins recognizing antigen T, that is, ABL (black), and PNA (gray). BSA was used as negative control. Tumor cells HT29 were marked with calcein-AM. The cell suspension marked with calcein was incubated with serum sample dilutions ( 1/10 or 1/20) for 1 hour at 37° C., and was then placed in the wells covered by proteins. The cells were left to adhere during 120 minutes at 37° C. Non-adherent cells marked with calcein were carefully removed with washing with RPMI. Fluorescence was measured with a fluorescent image reader FLA-3000 (Fujifilm). Anti-KLHs sera was used as reference for calculating adhesion percentage.

### DETAILED DESCRIPTION OF THE INVENTION

The present invention provides a T immunogen modified by glycane engineering, particularly a new topologic design of TFD is disclosed, which allows a greater control of the glycosidic link, in order to promote glycane immunogenicity, generating an immune response which recognizes antigen T present in tumors. The immunogen of the present invention has reduced flexibility, increasing immune response against antigen T, thus generating a therapeutic strategy for tumor treatment in oncologic patients, reducing malignant cells proliferation, and reducing metastases dissemination.

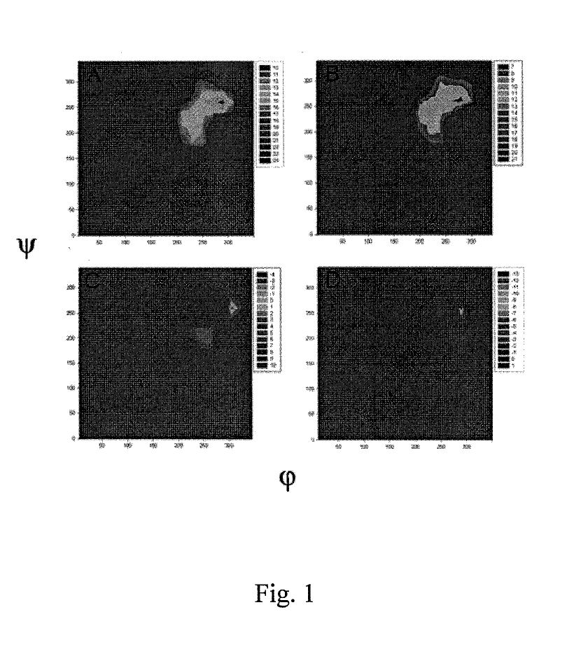

TFD rigidity is an important variable in controlling glycane immunogenic design. As shown in FIG. 1, a comparative analysis of antigen T glycosidic flexibility in MUC1 (GSTAP) in reference to a modified TFD topology was performed using an MM2 energy function program. These theory studies suggested that connector penta-lysine (Lys5), and a benzyl hydrophobic terminal tag (benzyl [Bzl] residue) were useful changes to reduce TFD flexibility. The contribution of each residue (connector Lys5 and residue Bzl) to TFD rigidity may be appreciated in the Ramachandran graphs of FIG. 1. Glycopeptide with both changes (BzlaTFD-Lys5) disclosed a significantly lower energy configuration (−13 kcal/mol) compared to MUC1 antigen T (10 kcal/mol). Valley Besides, BzlαTFD-Lys5 showed a clear reduction of the Gal-O-GalNAc glycosidic link energetic valley, compared to antigen T, generating the lesser amount of possible conformations for a similar energetic design, which increases its molecular rigidity. Maximal rigidity of glycosidic link Galβ3GalNAc was obtained using Lys5 as connector, and terminal residue Bzl. Connector Lys5 is itself the cause of TFD rigidity, but its combination with residue Bzl reduce even more glycane flexibility. Any person skilled in the art knows that by using a lysine connector to increase immunogen rigidity, a connector having an amount between 1 and 5 lysines may be used, thus any lysine connector falls within the scope of the present invention.

The procedure to obtain the invention immunogen was as follows:

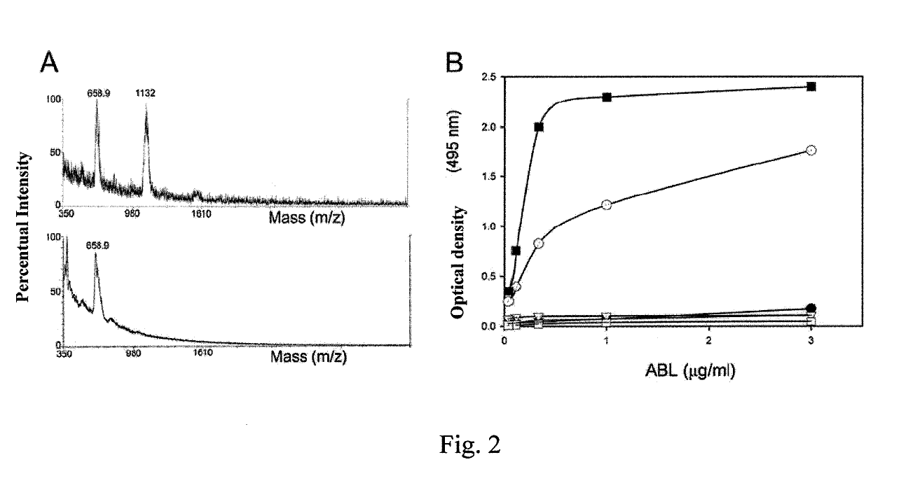

A) in a first stage, it was demonstrated that link BzlαTFD and Lys5 is possible to perform. In this function, the action of galactose oxydasa on BzlαTFD was used to generate an aldehyde in terminal galactose C6, which was covalently linked to linker Lys5 by reductive amination in the presence of NaCNBH₃. Here it was demonstrated that covalent link between BzlαTFD and Lys5 may be obtained in the described experimental conditions, and the link stoichiometry is one molecule of BzlαTFD for each Lys5, which is evidenced by the molecular weight obtained by mass spectrometry. Glyco-conjugate BzlαTFD-Lys5 molecular weight, determined by MALDI-TOF mass spectrometry, was 1132 Da (FIG. 2A)).

B) in a second stage, total procedure was carried out to obtain the invention immunogen, which is described as follows: connector Lys5 was conjugated to the carrier protein (KLHs) in the presence of 1-etil-3-(3-dimethylaminepropyl) carbodiimide (EDC). In parallel, BzlαTFD was oxydized in the terminal galactose C6 by using galactose oxydasa. Covalent link between aldehyde BzlαTFD Gal C6 and Lys5-KLHs amine residue was established by action of NaCNBH₃. The disaccharide covalent link to Lys5-KLHs is shown by the lectin ability to link to a carbohydrate. FIG. 2B shows the capacity of BzlαTFD-Lys5-KLHs to link to Agaricus bisporus lectin (ABL), a protein recognizing antigen T. Interaction with lectin shows the presence of disaccharide in the designed construction. By combining experiences, covalent link of BzlαTFD to Lys5-KLHs is shown, and thus obtaining the invention immunogen.

The procedure described in the previous paragraph is a way of obtaining and synthesis of the present invention immunogen, where such a procedure is one of the preferred embodiments. The persons skilled in the art know that other alternative procedures may be carried out without altering the spirit of the present invention.

In order to show the present invention immunogen immunogenic capacity, ALB/c mice were immunized with BzlαTFD-Lys5-KLHs invention immunogens, asyaloglycoforin(ASG)-KLHs, and KLHs. The immune response was studied by ELISA. Analyzed sera showed high IgG e IgM levels against the injected antigens, indicating an adequate choice of the animals and immunization protocol. No response was observed against bovine serum albumin (BSA), which was used as antigen control, (see Table 1). Immunizations with the invention immunogen (BzlαTFD-Lys5-KLHs) generated high levels of IgG and IgM type antibodies against BzlαTFD-BSA immunogen, in reference to anti-KLHs control antigen. These IgG and IgM antibodies also recognized TFD-BSA as antigen, indicating a significant anti-TFD immune response which is not exacerbated by using ASG as immunogen. This TDF humoral recognition is an important finding which suggest the possibility of linking tumoral antigen T. The use of BzlαTFD-Lys5-KLHs as antigen produced tumor anti-antigen T T-MUC1 antibody levels of 400 (IgG) and 1600 (IgM). The invention immunogen BzlαTFD-Lys5-KLHs generated higher anti-antigen T levels compared to the modified glycane with ASG, a glycoprotein with multiples O-glycosilation sites mainly exposing antigen T configurations. The immune response obtained by immunization with modified TFD immunogen did not show linking to glycolipids with Galβ3GalNAc exposed as terminal residue (Table 1). Antigens GA1, N5c, and N6 were not recognized by the antibodies here generated by ELISA o HPTLC immunostaining assays.

TABLE 1
Titration of antibodies measured by por ELISA^{a}

| | Immunogen | |
|---|---|---|
| Aalyzed | BzlαTFD-Lys5-KLHs | ASG-KLHs |

| antigen | IgG | IgM | IgG | IgM |
|---|---|---|---|---|
| BSA | 0^{b} | 0 | 0 | 0 |
| BzlαTFD- | >12800 | >12800 | 0 | 0 |
| BSA | | | | |
| TFD-BSA | 1600 | 3200 | 0 | 0 |
| MUC1 | 0 | 0 | 0 | 0 |
| T-MUC1 | 400 | 1600 | 50 | 50 |
| GA1^{c} | 0 | 0 | 0 | 0 |
| N5c^{d} | 0 | 0 | 0 | 0 |
| N6^{e} | 0 | 0 | 0 | 0 |

^{a}Concentration by ELISA was defined as the reverse of the highest dilution generating an absorbance value ≧0.01 referred to control serum. Control serum was obtained by immunizations with KLHs.
^{b}Value of 0 is a dilution <50
^{c}GA1 (Galβ3GalNAcβ4Galβ4Glcβ-Cer)
^{d}N5c (Galβ3GalNAcβ4GlcNAcβ3Manβ4Glcβ-Cer)
^{e}N6 (Galβ3GalNAcβ4GalNAcβ4GlcNAcβ3Manβ4Glcβ-Cer).

These antibodies were analyzed by competition ELISA to determine their glycidic recognition fine specificity. Inhibitory effect of TDF related disaccharides in the interaction of TFD-BSA/antigen T-IgG/IgM antibodies. BzlαGalNAc, BzlαTFD, and TFD showed significant inhibition, while Glc and GlcNAc did not show any effect (Table 2). Disaccharide specificity was reflected by the higher inhibitory effect of TFD compared to GalNAc or Gal. BzlαTFD was also better inhibitor than BzlαGalNAc where tumor antigen T or TFD-BSA were assessed as antigens, again demonstrating the importance of the disaccharide in glycidic recognition.

TABLE 2
Inhibition of antibody interaction using TDF related carbohydrates

| | Concentration (mM) required to produce 50% | | | |
|---|---|---|---|---|
| | inhibition (IC50) | | | |
| | TFD-BSA antigen | | T-MUC1 antigen | |
| Inhibitor | IgG | IgM | IgG | IgM |
| Glc | >100 | >100 | >100 | >100 |
| Gal | >100 | >100 | >100 | >100 |
| GlcNAc | >100 | >100 | >100 | >100 |
| GalNAc | 20 | 30 | >100 | >100 |
| BzlαGalNAc | 5 | 1 | 20 | >20 |
| TFD | 10 | 3.5 | 20 | >20 |
| BzlαTFD | 2.5 | 0.1 | 10 | >10 |

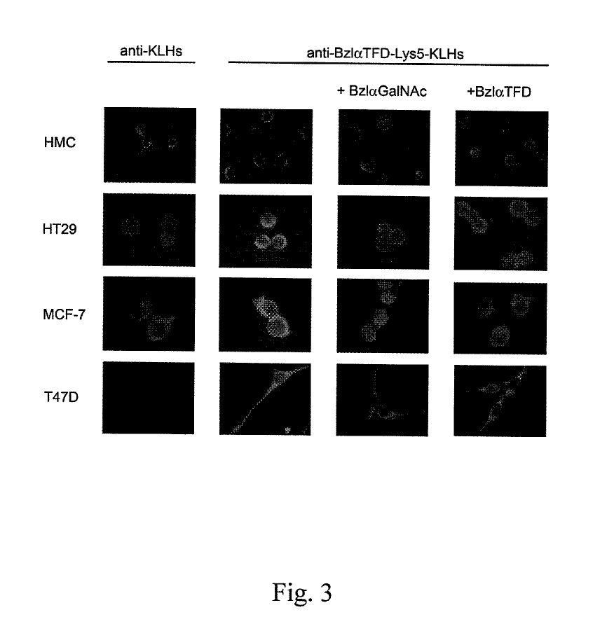

Reactivity of the antibodies generated by the invention immunogen BzlαTFD-Lys5-KLHs was studied in diverse epithelial tumor cell lines by cell immunocytology and ELISA. Breast human carcinoma cell lines such as T47D, and MCF7, and colon adenocarcinoma such as HT29 were used. In immunocytochemistry essays, tumor cells HT29, T47D, and MCF7 showed a clear positive tinction in reference to control serum, with a 1/100 dilution of sera used (FIG. 3), which indicates that antibodies so generated recognize antigen structures present in tumor cells. Cell ELISA assays also disclosed considerable antibody levels (Table 3). Results obtained by both methods showed that the invention immunogen BzlαTFD-Lys5-KLHs generates antibodies recognizing different human epithelial tumor lines (see Table 3).

TABLE 3
Levels of anti-tumoral cell antibodies measured by ELISA in cells^{a}

BzlαTFD-Lys5-KLHs
immunogen

| Tumor line | IgG | IgM |
|---|---|---|
| HT29 | 400 | 400 |
| MCF7 | 200 | 800 |
| T47D | 100 | 400 |

^{a}Level by ELISA of cells is defined as ELISA

Quantitative evidences demonstrated by competitive ELISA of cells disclosed that glycanes are antibody-tumor cell interaction targets. TFD related carbohydrate inhibition TFD demontrated glycane specificity of the antibodies generated by el BzlαTFD-Lys5-KLHs invention immunogen recognizing tumor cells. FIG. 3 and Table 4 show the inhibitory effects of TFD related molecules. In the IgM mediated interaction, BzlαTFD, and pNPαTFD were greatest inhibitors, TFD and BzlαGalNAc showed 4 times less inhibitory effect, GalNAc was 10 times lesser than BzlαTFD, and Gal, GlcNAc and Glc did not show inhibitory effect. Similar results were obtained for IgG type antibodies. This shows that antibodies generated linking to tumor cells are directed towards structures related to antigen T.

TABLE 4
Inhibition of antibodies with HT29 tumor cell interaction

Concentration (mM) requested to produce 50%
inhibition (IC50)

| Inhibitor | IgG | IgM |
|---|---|---|
| Glc | >100 | >100 |
| Gal | >100 | >100 |
| GlcNAc | >100 | >100 |
| GalNAc | >100 | 20 |
| BzlαGalNAc | 8 | 8 |
| TFD | >10 | 8 |
| BzlαTFD | 4 | 2 |
| pNPαTFD | >10 | 2 |

The present invention immunogen generates antibodies recognizing epithelial tumor cells. This feature indicates its potential application in the development of vaccines, active immunization in oncologic patients, and monoclonal antibodies technology based on primary stimulation.

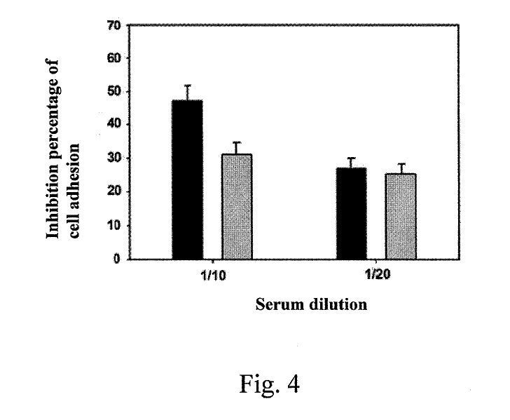

Over-expression of antigens T in tumors correlates with their metastatic potential. Lectins are proteins which recognize glycanes, and they are often involved in recognition and communication between cells. Antigen T plays an important role in tumor cell adhesion to lectins expressed by endothelial cells during metastatic dissemination. The ability of antibodies generated by the invention immunogen (BzlαTFD-Lys5-KLHs) to inhibit tumor cell adhesion to lectins recognizing antigen T was studied. FIG. 4 show the effect of these antibodies on tumor cell adhesion to lectins ABL and PNA. Anti-antigen T antibodies in 1/10 and 1/20 dilutions caused 47 and 25% reduction in tumor adhesion, respectively. These results shoes that antibodies generated by the present invention immunogen BzlαTFD-Lys5-KLHs, have potential therapeutic application in metastasis prevention.

Metastatic dissemination is a common way of epithelial tumor cells invading tissues. This dissemination occurs though the blood flow, with primary adhesion of tumor to endothelial cells mediated by lectin interaction. Since antibodies generated against the present invention immunogen (BzlαTFD-Lsy5-KLHs) reduced tumor cell adhesion to lectins recognizing antigen T, these antibodies could reduce metastatic dissemination, thus reducing tumor aggressiveness, improving patient prognosis.

Persons skilled in the art know that the invention immunogen may comprise any hydrophobic terminal residue linked to TFD disaccharide, a connector comprising an amount between 1 and 5 lysine amino acids, and any carrier which may link to the connector, with all configurations enclosed in the scope of present invention. In a preferred embodiment, the invention immunogen is of the following formula or configuration: BzlαTFD-Lsy5-KLHs.

Finally, the present invention immunogen may promote humoral immune response against tumor antigen T. Through this promoting effect of the antibody-tumor cell recognition, and the tumor cell adhesion inhibition effect, such immunogen provides a tool for cancer therapy and treatment, reduction of metastasis, and increase of anti-T immunity.

It is highly relevant to highlight that the immunogen described in this invention patent application may generate an immune response which recognizes tumor antigen T (T-MUC1), a result not achieved/described in previous immunogen disclosures.

This invention is better illustrated according to the following examples, which must not be construed as a limititation to its scope. On the contrary, it must clearly be understood that other embodiments, modifications, and equivalents thereof may be referred to after reading this description, which may suggest the skilled person in the art without leaving the spirit of the present invention and/or the scope of the annexed claims.

### EXAMPLES

### Example 1

### Molecular Conformations

Initial structures of glycoconjugates (antigen T, BzlαTFD, TFD-Lys5, and BzlαTFD-Lys5) were generated by a molecular editor. Conformations of glycoconjugates were obtained by minimization of structure energy using a MM2 energy function. Ramachandran diagrams were constructed by the conformer energy during rotation of torsion angles φ and ψ of Gal-O-GalNAc (FIG. 1).

### Example 2

### Covalente Link Between de BzlαTFD and Lys5

To obtain 1 ml oxydized BzlαTFD in C6 (4 mM) 50 U galactose oxydase imobilized in Sepharose 4B gel activated with cyanogens bromide was obtained. The carbohydrate was recovered by gel separation, such carbohydrate oxidation was controlled, it was mixed with 3 mg Lys5 in PBS for 10 minutes, and 3 mg NaCNBH₃ were added. Reaction was incubated for 4 hours at room temperature, and agitating. Molecular weights of glycoconjugate and nude peptide were determined by MALDI-TOF mass spectrometry over a Voyager-DE™ Pro Biospectrometry Workstation from Applied Biosystems (FIG. 2A).

### Example 3

### Construction of Manipulated TFD Immunogen

Connector Lys5 was conjugated to KLHs using 1-ethyl-3-(3-dimethylaminopropyl) carbodiimide (EDC) as crossing agent. 7.5 mg KLHs were mixed in 400 μl sodium phosphate buffer (50 mM pH 4.7) with 6 mg EDC for 1 hour at 4° C., then 100 μl Lys5 (50 mM) were added in water adjusting reaction pH at 5 with 50 mM sodium phosphate buffer, and it was incubated at 4° C. for 20 hours. Sample was dialyzed with four changes of sodium phosphate buffer (10 mM, pH 7.2) with 150 mM sodium chloride (PBS) at 4° C. Finally, 200 μl oxydized BzlαTFD was incubated in C6 (4 mM) with Lys5-KLHs for 10 minutes, and then 10 mg de NaCNBH₃ were added. Reaction mixture was incubated with agitation for 4 hours at room temperature, and then overnight at 4° C. Immunogen BzlαTFD-Lys5-KLHs was extensively dialyzed against PBS and kept at −20° C. until used as immunogen. As immunogen for control serum, KLHs without additional treatment was used. ASG was used as immunogen having exposed antigen T. Glycoconjugation control was based on the lectin inability to recognize glycanes (FIG. 2). Agaricus bisporus lectin reactivity with glycoconjugates was analyzed by enzyme-lectin assay (ELA). Multi-well polystirene plates were activated with different antigens (5 μg/ml) in PBS overnight at 4° C., then incubated with blocking buffer, followed by incubation with diverse ABL-HRP concentrations foe 2 hours at room temperature, and six washings with PBS. Color reaction was developed using 2 mg/ml o-phenylenediamine and H₂O₂ 0.02% in sodium citrate buffer, pH 5 for 20 minutes at room temperature. Reaction was stopped by adding 2.5 M sulfuric acid, and absorbance values were read at 490 nm using a plate reader.

### Example 4

### Mice Immunization

Groups of 5 mice (BALB/c female, 6 weeks old) were intradermally injected with 100 μg invention immunogen mixed with 100 μl complete Freund's adjuvant, in for shaved sites of the mice. After two weeks, this process was repeated, but using incomplete Freund's adjuvant. Two weeks later, a boost was administered subcutaneously with the same sample used in the second inoculation. The mice were bleeded on day 0 and 10 days after the third immunization.

### Example 5

### Gycoconjugated Immunogens

2 mM BzlαTFD or oxydized TFD in C6 (1 ml), obtained by treatment with galactose oxydase, were mixed with 3 mg BSA in PBS for 10 minutes, and then 3 mg NaCNBH₃ were added. The reaction mixture was incubated with agitation for 4 hours at room temperature. The glycoconjugate was dialyzed with four changes of PBS at 4° C. The presence of carbohydrate linked to proteins was analyzed by ELA.

The chemioenzymatic O-glycoconjugated MUC1 was prepared using a human synthetic MUC1 of 60 mer (3 repetitions in groups of 20 amino acids: HGVTSAPDTRPAPGSTAPPA), and recombinant GalNAc-T2 and -T4 transferases, obtaining 15×GalNAc O-glycane by tandem repetition of MUC1 (Hanisch F G. et al., Glycobiology 11, 731-740, 2001). Glycopeptide GalNAcαMUC1 was purified by HPLC using a reverse phase column and incubated with dC1Gal-T1 (Core 1 sinthase), a glycoprotein-N-acetylgalactosamine 3-β-galactosetransferase from Drosophila melanogaster (Ju T. et al J. Biol. Chem. 277, 178-86, 2002) (EC 2.4.1.122), for the construction of Galβ3GalNAcαMUC1 (T-MUC1). After purifying by HPLC, the glycoconjugate sample was checked by MALDI-TOF MS.

### Example 6

### Enzyme Linked Immunosorption Assay (ELISA) and Competition ELISA

Target antigens (10 μg/ml) were adhered to multi-well plates of polystirene in saline phosphate buffer (PBS; 10 mM sodium phosphate, pH 7.2 and 150 mM NaCl) overnight at 4° C. Glycolipids were diluted in methanol and dried for 1 hour at 60° C. After covering the wells with the antigens, these were saturated with PBS-1% BSA-0.05% Tween 20 for 1 hour at 37° C. Serial dilutions of antiserum were incubates with the antigen for 2 hours at room temperature, and then washed 5 times with PBS. The adhered antigen-antibody complex was detected with a 1/1000 dilution of anti-IgG goat antibody or mouse IgM conjugated with peroxydase in PBS-0.05% Tween 20 (PBS-T), incubates at room temperature for 1 hour, and washed 5 times with PBS. Color reaction was developed as described for ELA.

The three antibodies showing highest anti-TFD and anti-antigen T levels were analyzed by competition ELISA, and results were averaged. Optimum antiserum dilutions with an optical density of 1.0 against TFD-BSA and T-MUC1 (10 μg/ml) as blanc antigen were determined in preliminary experiments. Competition ELISA stages were equal to those in ELISA, except in that optimal antiserum dilution was pre-incubated with several related antigens in carbohydrates for 1 hour at room temperature before being added to the wells. Minimum concentration of carbohydrates required for 50% inhibition (ID50) of antigen-antibody interaction is a more specific parameter.

### Example 7

### HPTLC Immunotinction

Glycolipids were separated in HPTLC silica gel 60 in the chlorform/methanol/aqueous run solvent 0.2% CaCl₂ (45:45:10), using a tank, in order to obtain highly reproducible chromatograms. Plates were dried in air for 15 minutes, covered by immersion in a poly-isobutylmethacrilate 0.5% in hexane/chloroform (9:1) for 1 minute, dried again in air for 10 minutes, incubated with a 1/50 serum dilution in PBS overnight at 4° C., washed with PBS, incubated with a 1/1000 dilution of mice anti-Ig goat antibody conjugated with peroxydase in PBS at room temperature for 1 hour, and washed 5 times with PBS. Color reaction was developed using 4-chloro-1-naftol 0.1 mg/ml and H₂O₂ 0.02% in metanol-PBS (1:29) for 20 minutes, and stopped by washing with distilled water.

### Example 8

### Marking of Tumor Cells

Immunocytology was developed using breast carcinoma human cell lines T47D and MCF7, and colon adenocarcinoma HT29, cultured to sub-confluence in appropriate media recommended by the American Type Culture Collection (ATCC). The cells were fixed on slides covered with polylysine using methanol at −20° C. for 10 minutes, then they were blocked with BSA 1% in PBS and sera were diluted in PBS and incubated with the cells for 2 hours at 4° C. Linked antibodies were detected with a mouse anti-IgG rabbit antibody 1/1000 dilution conjugated to Alexa488 (Molecular Probes). Slides were washed with PBS and water, mounted with Fluor Save, and examined with a Zeiss fluorescence microscope. Steps for competitive assays were the same as for immunocytology, except in that the optimum anti-sera dilution was pre-incubated with different antigens related to carbohydrates for 1 hour at room temperature before being offered to the cells.

### Example 9

### ELISA of Cells and Competition ELISA of Cells

Level of antibodies against epithelial tumor cells was determined using lines HT29, MCF7, and T47D. Tumor cells were cultured to sub-comfluence in media recommended by ATCC, and harvested by treatment with trypsin. They were adsorbed to multi-well plates, fixed with cold methanol for minutes and saturated with PBS-T for 1 hour at room temperature. ELISA of cells was carried out by incubating serial dilutions of mouse sera against the different tumor lines for 2 hours at room temperature in PBS, and then was washed 4 times with PBS-T. The adhered antigen-antibody complex was detected with a 1/1000 dilution of anti-IgG goat antibody or mouse IgM conjugated to a peroxidase in PBS-T, incubated for 1 hour at room temperature, and washed times con PBS-T. Color reaction was developed as described for ELA. Steps for competition ELISA of cells were the same as for ELISA of cells, except in that the optimum anti-sera dilution was pre-incubated with several antigens related to carbohydrates for 1 hour at room temperature prior to the addition to the plate wells.

### Example 10

### Tumor Cell Adhesion Assay

This assay was designed to count cells adhered to multi-well plates, with a minimum experimental error. Multi-well plates were covered with ABL, PNA o BSA in sodium carbonate buffer 0.1 M, pH 9.5 overnight at 4° C., then 1 h at 37° C., followed by blocking buffer (PBS-Tween 20 0.1%) in order to prevent non-specific adsorption to the surface. Tumor cells were separated from the culture plates by a non-enzymatic treatment with 2 mM EDTA in PBS, treated with 50 mU/ml neuraminidase IV from Clostridium perfringens (Sigma) for 1 h at 37° C., washed twice with RPMI medium free from serum and re-suspended in 5×10⁶ cells/ml. A calcein-AM (Molecular Probe) solution was added to the cell suspension (5 μl per ml) to a final concentration of 5 μM. The cell suspension was mixed and incubated for 30 min at 37° C. in the dark. The calcein marked cell suspension (100 μl) was incubated with sera sample dilutions for 1 hour at 37° C., then placed in the wells covered with proteins, and the cells were allowed to adhere for 120 minutes at 37° C., in the dark. At the end of the incubation, the non-adhered cells marked with calcein were carefully removed by carefully washings (4 times) with 200 μl preheated RPMI per well. Fluorescence was analyzed with a FLA-3000 fluorescent image reader (Fujifilm). Results, after substracting non-specific adsorption (obtained by linking with BSA), were expressed as average percentage of cells linked in three to eight replicate experiments ±standard error. Anti-KLHs sera was used as reference for calculating adhesion percentage.

1. A modified Thomsen-Friedenreich disaccharide (TFD) immunogen, characterized by comprising general formula D-TFD-Lys(n)-C, wherein
D is a hydrophobic terminal residue,
TFD is disaccharide Galβ3GalNAcα;
Lys(n) is a lysine connector and n is an integer between 1 and 5, and
C is a carrier.

2. The immunogen of claim 1, characterized in that the hydrophobic terminal residue is selected from the group consisting of benzyl, p-nitrophenyl, butyl, propyl, ethyl, and methyl.

3. The immunogen of claim 2, characterized in that the hydrophobic terminal residue is benzyl.

4. The immunogen of claim 1, characterized in that the hydrophobic terminal residue is covalently linked to TFD GalNAc C1 in α position.

5. The immunogen of claim 1, characterized in that the lysine connector is a penta-lysine.

6. The immunogen of claim 1, characterized in that the carrier is selected from the group consisting of Keyhole limpets hemocyanin (KLH), and muramyl-dipeptide (MDP).

7. The immunogen of claim 6, characterized in that the carrier is Keyhole limpets hemocyanin.

8. The immunogen of claim 6, characterized in that the carrier is succinikated Keyhole limpets hemocyanin (KLHs).

9. The immunogen of claim 1, characterized by being BzlαTFD-Lys5-KLHs, wherein Bzl is benzyl, TFD is disaccharide Galβ3GalNAcα; Lys5 is a penta-lysine, and KLHs is succinilated Keyhole limpets hemocyanin.

10. A procedure for preparing immunogen of claim 1, characterized by comprising the following stages,
a. covalently link lysine connector Lys(n) to a carrier, wherein n is an integer between 1 and 5;
b. oxydize molecule BzlαTFD in terminal galactose carbon 6 to generate an aldehyde on said C6;
c. covalently link amine residue of connector Lys(n)-C from stage a) and aldehyde in terminal galactose C6 of BzIaTFD from stage de la b) by reduction amination in the presence of NaCNBH₃; y
d) dialyze the reaction product and recovery the immunogen.

11. The procedure of claim 10, characterized in that carrier C is selected from the group consisting of KLHs and muramyl-dipeptide (MDP).

12. The procedure of claim 10, characterized in that the carrier is KLHs and it links covalently to connector Lys(n) in the presence of 1-ethyl-3-(3-dimetilaminopropil) carbodiimida (EDC).

13. The procedure of claim 10, characterized in that the oxydation stage b) is carried out in the presence of the enzyme galactose oxydase.

14. A composition to increase anti-tumoral immunity, characterized by comprising an effective amount of a modified Thomsen-Friedenreich disaccharide (TDF) immunogen of formula D-TFD-Lys(n)-C, wherein D is a hydrophobic terminal residue, TFD is disaccharide Galβ3GalNAcα, Lys(n) is a lysine connector, and n is an integer between 1 and 5, and C is a carrier; and pharmaceutically acceptable supports and/or excipients.

15. The composition of claim 14, characterized in that the hydrophobic terminal residue is selected from the group consisting of benzyl, p-nitrophenyl, butyl, propyl, ethyl, and methyl.

16. The composition of claim 15, characterized in that the hydrophobic terminal residue is benzyl.

17. The composition of claim 14, characterized in that the hydrophobic terminal residue is covalently linked to TFD GalNAc C1 in position α.

18. The composition of claim 14, characterized in that the lysine connector is a penta-lysine.

19. The composition of claim 14, characterized in that the carrier is selected from the group consisting of Keyhole limpets hemocyanin (KLH), and muramyl-dipeptide (MDP).

20. The composition of claim 19, characterized in that the carrier is Keyhole limpets hemocyanin.

21. The composition of claim 19, characterized in that the carrier is succinilated Keyhole limpets hemocyanin.

22. The composition of claim 14, characterized in that the immunogen is BzlαTFD-Lys5-KLHs, wherein Bzl is benzyl, TFD is disaccharide Galβ3GalNAcα; Lys5 is a penta-lysine, and KLHs is succinilated Keyhole limpets hemocyanin.

23. The composition of claim 14, characterized by being in the form selected from the group consisting of liquid, solid, gels, and aerosol forms.

24. The composition of claim 14, characterized by also comprising an adjuvant.

25. A composition to inhibit adhesion of tumor cells and metastatic ability, characterized by comprising an effective amount of a modified Thomsen-Friedenreich disaccharide (TDF) of general formula D-TFD-Lys(n)-C, wherein D is a hydrophobic terminal residue, TFD is disaccharide Galβ3GalNAcα, Lys(n) is a lysine connector, and n is an integer between 1 and 5, and C is a carrier; and pharmaceutically acceptable supports and/or excipients.

26. The composition of claim 25, characterized in that the hydrophobic terminal residue is selected from the group consisting of benzyl, p-nitrofenilo, butyl, propyl, ethyl, and methyl.

27. The composition of claim 26, characterized in that the hydrophobic terminal residue is benzyl.

28. The composition of claim 25, characterized in that the hydrophobic terminal residue is covalently linked to TFD GalNAc C1 in position α.

29. The composition of claim 25, characterized in that the lysine connector is a penta-lysine.

30. The composition of claim 25, characterized in that the carrier is selected from the group consisting of Keyhole limpets hemocyanin (KLH), and muramyl dipeptide (MDP).

31. The composition of claim 30, characterized in that the carrier is Keyhole limpets hemocyanin.

32. The composition of claim 30, characterized in that the carrier is succinilated Keyhole limpets hemocyanin.

33. The composition of claim 25, characterized in that immunogen is BzlαTFD-Lys5-KLHs, wherein Bzl is benzyl, TFD is disaccharide Galβ3GalNAcα; Lys5 is a penta-lysine and KLHs is succinilated Keyhole limpets hemocyanin.

34. The composition of claim 25, characterized by being in the form selected from the group consisting in liquid, solid, gel, and aerosol forms.

35. The composition of claim 25, characterized by also comprising an adjuvant.

36. The use of the immunogen of claim 1 to prepare a medication to improve anti-tumor immunity.

37. The use of the immunogen of claim 1 to prepare a medication for the treatment of cancer.

38. The use of the immunogen of claim 1 to prepare a medication to reduce metastatic ability of a tumor.

39. A method for the immunization of a mammal against antigen T, characterized by this method comprising the administration of an effective amount of a composition, wherein said composition comprises a modified Thomsen-Friedenreich disaccharide (TDF) immunogen of general formula D-TFD-Lys(n)-C, wherein D is a hydrophobic terminal residue, TFD is disaccharide Galβ3GalNAcα, Lys(n) is a lysine connector, and n is an integer between 1 and 5, and C is a carrier; and pharmaceutically acceptable supports and/or excipients.

40. The method of claim 39, characterized in that the hydrophobic terminal residue is selected from the group consisting of benzyl, p-nitrophenyl, butyl, propyl, ethyl, and methyl.

41. The method of claim 40, characterized in that the hydrophobic terminal residue is benzyl.

42. The method of claim 39, characterized in that the hydrophobic terminal residue is covalently linked to TFD GalNAc C1 of position a.

43. The method of claim 39, characterized in that the lysine connector is a penta-lysine.

44. The method of claim 39, characterized in that carrier is selected from the group consisting of Keyhole limpets hemocyanin (KLH), and muramyl-dipeptide (MDP).

45. The method of claim 44, characterized in that the carrier is Keyhole limpets hemocyanin.

46. The method of claim 44, characterized in that the carrier is succinilated Keyhole limpets hemocyanin.

47. The method of claim 39, characterized in that the immunogen is BzlαTFD-Lys5-KLHs, wherein Bzl is benzyl, TFD is disaccharide Galβ3GalNAcα; Lys5 is a penta-lysine, and KLHs is succinilated Keyhole limpets hemocyanin.

48. The method of claim 39, characterized in that the mammal is a cancer carrier.

49. The method of claim 48, characterized in that the cancer is selected from the group consisting of breast cancer and colon cancer.

50. The method of claim 39, characterized in that the immunogen is applied jointly with substances increasing immunity.

51. The method of claim 50, characterized in that the substances increasing immunity are selected from the group consisting of cytokines and adjuvants.
